(19) United States
(12) Patent Application Publication
(10) Pub. No.: US 2026/0165598 A1
(43) Pub. Date: Jun. 18, 2026

(54) GENERATION OF ARTIFICIAL CONTRAST-ENHANCED RADIOLOGICAL IMAGES

(71) Applicant: BAYER AKTIENGESELLSCHAFT, Leverkusen (DE)

(72) Inventors: Gesine KNOBLOCH, Berlin (DE); Christian LIENERTH, Berlin (DE)

(21) Appl. No.: 19/128,649

(22) PCT Filed: Nov. 10, 2023
(86) PCT No.: PCT/EP2023/081378

§ 371 Date: May 9, 2025

(30) Foreign Application Priority Data
Nov. 12, 2022 (EP) 22207079.9

(51) Int. Cl. A61B 5/055; G01R 33/28; G01R 33/56; G06T 12/30

(52) U.S. Cl. CPC A61B 5/055; G01R 33/281; G01R 33/5601; G06T 12/30; G06T 2200/24; G06T 2210/41; G06T 2211/412

(57) ABSTRACT

The present invention relates to the technical field of generating artificial contrast-enhanced radiological images.

The present disclosure is concerned with the technical field of generation of artificial contrast-enhanced radiological images.

WO2019/074938A1 discloses a method for reducing the amount of contrast agent in the generation of radiological images with the aid of an artificial neural network.

In the disclosed method, a training data set is created in a first step. The training data set comprises, for a large number of persons and for each person, i) a native radiological image (zero-contrast image), ii) a radiological image after administration of a low amount of contrast agent (low-contrast image) and iii) a radiological image after administration of a standard amount of contrast agent (full-contrast image).

In a second step, an artificial neural network is trained to predict for each person of the training data set, on the basis of the native image and the image after administration of a low amount of contrast agent, an artificial radiological image showing an acquisition region after administration of the standard amount of contrast agent. The measured radiological image after administration of a standard amount of contrast agent serves in each case as reference (ground truth) in the training.

In a third step, the trained artificial neural network can be used to predict for a new person, on the basis of a native image and of a radiological image after administration of a low amount of contrast agent, an artificial radiological image which shows the acquired region as it would look if a standard amount of contrast agent had been administered.

The method disclosed in WO2019/074938A1 has disadvantages.

For instance, training data are required for training the artificial neural network. A large number of radiological examinations need to be carried out on a large number of persons and the training data need to be generated in order to be able to train the network.

The artificial neural network disclosed in WO2019/074938A1 is trained to predict a radiological image after administration of a standard amount of a contrast agent. The artificial neural network is not configured and not trained to predict a radiological image after administration of an amount lower or higher than the standard amount of contrast agent. The method described in WO2019/074938A1 can in principle be trained to predict a radiological image after administration of an amount of contrast agent different than the standard amount. However, this requires further training data and further training.

It would be desirable to be able to generate radiological images with variable contrast enhancement without needing to generate training data for each individual contrast enhancement and without needing to train an artificial neural network. It would additionally be desirable to be able to generate radiological images with variable contrast enhancement using a reproducible deterministic process to generate the variable contrast enhancement. This facilitates the approval and use of a corresponding medical procedure, while minimizing false negative and false positive results. Machine learning methods employ statistical models that are of limited generalizability because they are usually based on a limited selection of training data. It would additionally be desirable to be able to generate radiological images with variable contrast enhancement using a wide variety of contrast agents. It would additionally be desirable to be able to use the method for generating radiological images with variable contrast enhancement using a wide variety of different contrast agents irrespective of their physical, chemical, physiological or other properties.

These and other objects are achieved by the subject matter of the independent claims. Preferred embodiments of the present disclosure are found in the dependent claims, in the present description and in the drawings.

The present disclosure thus provides in a first aspect a computer-implemented method for generating a contrast-enhanced radiological image, comprising the steps of:
receiving a first representation of an examination region of an examination object, the first representation representing the examination region without contrast agent or after administration of a first amount of a contrast agent,
receiving a second representation, the second representation representing the examination region of the examination object after administration of a second amount of the contrast agent, the second amount being larger than the first amount,
generating a third representation on the basis of the first representation and the second representation, the generating the third representation comprising subtracting the first representation from the second representation,
generating a fourth representation, the generating the fourth representation comprising adding the third representation to the first representation α times, α being a positive or negative real number,
outputting and/or storing the fourth representation and/or transmitting the fourth representation to a separate computer system.

The present disclosure further provides a computer system comprising:
a processor; and
a memory that stores an application program configured to perform an operation when executed by the processor, said operation comprising:
   receiving a first representation of an examination region of an examination object, the first representation representing the examination region without contrast agent or after administration of a first amount of a contrast agent,
   receiving a second representation, the second representation representing the examination region of the examination object after administration of a second amount of the contrast agent, the second amount being larger than the first amount,
   generating a third representation on the basis of the first representation and the second representation, the generating the third representation comprising subtracting the first representation from the second representation,
   generating a fourth representation, the generating the fourth representation comprising adding the third representation to the first representation α times, α being a positive or negative real number,
   outputting and/or storing the fourth representation and/or transmitting the fourth representation to a separate computer system.

The present disclosure further provides a computer program that can be loaded into a working memory of a computer system, where it causes the computer system to execute the following steps:
receiving a first representation of an examination region of an examination object, the first representation representing the examination region without contrast agent or after administration of a first amount of a contrast agent,
receiving a second representation, the second representation representing the examination region of the examination object after administration of a second amount of the contrast agent, the second amount being larger than the first amount,
generating a third representation on the basis of the first representation and the second representation, the generating the third representation comprising subtracting the first representation from the second representation,
generating a fourth representation, the generating the fourth representation comprising adding the third representation to the first representation α times, α being a positive or negative real number,
outputting and/or storing the fourth representation and/or transmitting the fourth representation to a separate computer system.

The present disclosure further provides for a use of a contrast agent in a radiological examination comprising:
receiving or generating a first representation of an examination region of an examination object, the first representation representing the examination region without the contrast agent or after administration of a first amount of the contrast agent,
receiving or generating a second representation, the second representation representing the examination region of the examination object after administration of a second amount of the contrast agent, the second amount being larger than the first amount,
generating a third representation on the basis of the first representation and the second representation, the generating the third representation comprising subtracting the first representation from the second representation,
generating a fourth representation, the generating the fourth representation comprising adding the third representation to the first representation α times, α being a positive or negative real number,
outputting and/or storing the fourth representation and/or transmitting the fourth representation to a separate computer system.

The present disclosure further provides a contrast agent for use in a radiological examination method comprising:
receiving or generating a first representation of an examination region of an examination object, the first representation representing the examination region without the contrast agent or after administration of a first amount of the contrast agent,
receiving or generating a second representation, the second representation representing the examination region of the examination object after administration of a second amount of the contrast agent, the second amount being larger than the first amount,
generating a third representation on the basis of the first representation and the second representation, the generating the third representation comprising subtracting the first representation from the second representation,
generating a fourth representation, the generating the fourth representation comprising adding the third representation to the first representation α times, α being a positive or negative real number,
outputting and/or storing the fourth representation and/or transmitting the fourth representation to a separate computer system.

The present disclosure further provides a kit comprising a computer program product and a contrast agent, the computer program product comprising a computer program that can be loaded into a working memory of a computer system, where it causes the computer system to execute the following steps:
receiving a first representation of an examination region of an examination object, the first representation representing the examination region without the contrast agent or after administration of a first amount of the contrast agent,
receiving a second representation, the second representation representing the examination region of the examination object after administration of a second amount of the contrast agent, the second amount being larger than the first amount,
generating a third representation on the basis of the first representation and the second representation, the generating the third representation comprising subtracting the first representation from the second representation,
generating a fourth representation, the generating the fourth representation comprising adding the third representation to the first representation α times, α being a positive or negative real number,
outputting and/or storing the fourth representation and/or transmitting the fourth representation to a separate computer system.

The subjects of the present disclosure will be more particularly elucidated below, without distinguishing between the subjects (method, computer system, computer program (product), use, contrast agent for use, kit). Rather, the elucidations that follow are intended to apply by analogy to all subjects, irrespective of the context (method, computer system, computer program (product), use, contrast agent for use, kit) in which they occur.

Where steps are stated in an order in the present description or in the claims, this does not necessarily mean that this disclosure is limited to the order stated. Instead, it is conceivable that the steps are also executed in a different order or else in parallel with one another, the exception being when one step builds on another step, thereby making it imperative that the step building on the previous step be executed next (which will however become clear in the individual case). The stated orders thus constitute preferred embodiments.

The present disclosure describes means which, on the basis of at least two representations representing an examination region of an examination object after addition/administration/use of different amounts of contrast agent, generate one or more artificial radiological images in which the contrast between areas with contrast agent and areas without contrast agent can be varied.

The “examination object” is normally a living being, preferably a mammal, most preferably a human.

The “examination region” is a part of the examination object, for example an organ or part of an organ or a plurality of organs or another part of the examination object.

For example, the examination region may be a liver, kidney, heart, lung, brain, stomach, bladder, prostate, intestine or a part thereof or another part of the body of a mammal (for example a human).

In one embodiment, the examination region includes a liver or part of a liver or the examination region is a liver or part of a liver of a mammal, preferably a human.

In a further embodiment, the examination region includes a brain or part of a brain or the examination region is a brain or part of a brain of a mammal, preferably a human.

In a further embodiment, the examination region includes a heart or part of a heart or the examination region is a heart or part of a heart of a mammal, preferably a human.

In a further embodiment, the examination region includes a thorax or part of a thorax or the examination region is a thorax or part of a thorax of a mammal, preferably a human.

In a further embodiment, the examination region includes a stomach or part of a stomach or the examination region is a stomach or part of a stomach of a mammal, preferably a human.

In a further embodiment, the examination region includes a pancreas or part of a pancreas or the examination region is a pancreas or part of a pancreas of a mammal, preferably a human.

In a further embodiment, the examination region includes a kidney or part of a kidney or the examination region is a kidney or part of a kidney of a mammal, preferably a human.

In a further embodiment, the examination region includes one or both lungs or part of a lung of a mammal, preferably a human.

In a further embodiment, the examination region includes a breast or part of a breast or the examination region is a breast or part of a breast of a female mammal, preferably a female human.

In a further embodiment, the examination region includes a prostate or part of a prostate or the examination region is a prostate or part of a prostate of a male mammal, preferably a male human.

The examination region, also referred to as the field of view (FOV), is in particular a volume that is imaged in radiological images. The examination region is typically defined by a radiologist, for example on a localizer image. It is of course also possible for the examination region to be alternatively or additionally defined in an automated manner, for example on the basis of a selected protocol.

The examination region is subjected to a radiological examination.

“Radiology” is the branch of medicine that is concerned with the use of electromagnetic rays and mechanical waves (including for instance ultrasound diagnostics) for diagnostic, therapeutic and/or scientific purposes. Besides X-rays, other ionizing radiation such as gamma radiation or electrons are also used. Imaging being a key application, other imaging methods such as sonography and magnetic resonance imaging (nuclear magnetic resonance imaging) are also counted as radiology, even though no ionizing radiation is used in these methods. The term “radiology” in the context of the present disclosure thus encompasses in particular the following examination methods: computed tomography, magnetic resonance imaging, sonography.

In one embodiment of the present disclosure, the radiological examination is a magnetic resonance imaging examination.

In a further embodiment, the radiological examination is a computed tomography examination.

In one embodiment, the radiological examination is an ultrasound examination.

In radiological examinations, contrast agents are commonly used for contrast enhancement.

“Contrast agents” are substances or mixtures of substances that improve the depiction of structures and functions of the body in radiological examinations.

In computed tomography, iodine-containing solutions are normally used as contrast agents. In magnetic resonance imaging (MRI), superparamagnetic substances (e.g. iron oxide nanoparticles, superparamagnetic iron-platinum particles (SIPPs)) or paramagnetic substances (e.g. gadolinium chelates, manganese chelates) are usually used as contrast agents. In the case of sonography, liquids containing gas-filled microbubbles are usually administered intravenously. Examples of contrast agents can be found in the literature (see for example A. S. L. Jascinth et al.: Contrast Agents in computed tomography: A Review, Journal of Applied Dental and Medical Sciences, 2016, vol. 2, issue 2, 143-149; H. Lusic et al.: X-ray-Computed Tomography Contrast Agents, Chem. Rev. 2013, 113, 3, 1641-1666; https://www.radiology.wisc.edu/wp-content/uploads/2017/10/contrast-agents-tutorial.pdf, M. R. Nouh et al.: Radiographic and magnetic resonances contrast agents: Essentials and tips for safe practices, World J Radiol. 2017 Sep. 28; 9(9): 339-349; L. C. Abonyi et al.: Intravascular Contrast Media in Radiography: Historical Development & Review of Risk Factors for Adverse Reactions, South American Journal of Clinical Research, 2016, vol. 3, issue 1, 1-10; ACR Manual on Contrast Media, 2020, ISBN: 978-1-55903-012-0; A. Ignee et al.: Ultrasound contrast agents, Endosc Ultrasound. 2016 November-December; 5(6): 355-362).

MRI contrast agents exert their effect by altering the relaxation times of structures that take up contrast agents. A distinction can be made between two groups of substances: paramagnetic and superparamagnetic substances. Both groups of substances have unpaired electrons that induce a magnetic field around the individual atoms or molecules. Superparamagnetic contrast agents result in a predominant shortening of T2, whereas paramagnetic contrast agents mainly result in a shortening of T1. The effect of said contrast agents is indirect, since the contrast agent does not itself emit a signal, but instead merely influences the intensity of signals in its vicinity. An example of a superparamagnetic contrast agent is iron oxide nanoparticles (SPIO, superparamagnetic iron oxide). Examples of paramagnetic contrast agents are gadolinium chelates such as gadopentetate dimeglumine (trade name: Magnevist® and others), gadoteric acid (Dotarem®, Dotagita®, Cyclolux®), gadodiamide (Omniscan®), gadoteridol (ProHance®), gadobutrol (Gadovist®), and gadoxetic acid (Primovist®/Eovist®).

The generation of an artificial radiological image with variable contrast enhancement is based on at least two representations of the examination region, a first representation and a second representation.

The first representation represents the examination region without contrast agent or after administration of a first amount of a contrast agent. Preferably, the first representation represents the examination region without contrast agent.

The second representation represents the examination region after administration of a second amount of a contrast agent. The second amount is larger than the first amount (which, as described, may also be zero). The expression “after the second amount of a contrast agent” should not be understood as meaning that the first amount and the second amount add up in the examination region (unless the first amount is zero). Thus, the expression “the representation represents the examination region after administration of a (first or second) amount” should rather be understood as meaning: “the representation represents the examination region with a (first or second) amount” or “the representation represents the examination region including a (first or second) amount”.

If the first amount is not equal to zero, then the first representation and the second representation preferably (but not necessarily) represent the examination region at the same time interval from the administration of the contrast agent. If the first amount is equal to zero, the time interval from the moment of administration of the contrast agent may be chosen as desired for the second representation.

Preferably, both the first amount and the second amount of the contrast agent are smaller than the standard amount.

The standard amount is normally the amount recommended by the manufacturer and/or distributor of the contrast agent and/or the amount authorized by a regulatory authority and/or the amount specified in a package leaflet for the contrast agent.

For example, the standard amount of Primovist® is thus 0.025 mmol Gd-EOB-DTPA disodium/kg body weight.

In one embodiment of the present disclosure, the contrast agent is an agent that includes gadolinium(III) 2-[4,7,10-tris(carboxymethyl)-1,4,7,10-tetrazacyclododec-1-yl]acetic acid (also referred to as gadolinium-DOTA or gadoteric acid).

In a further embodiment of the present disclosure, the contrast agent is an agent that includes gadolinium(III) ethoxybenzyldiethylenetriaminepentaacetic acid (Gd-EOB-DTPA); preferably, the contrast agent includes the disodium salt of gadolinium(III) ethoxybenzyldiethylenetriaminepentaacetic acid (also referred to as gadoxetic acid).

In one embodiment of the present disclosure, the contrast agent is an agent that includes gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate (also referred to as gadopiclenol) (see for example WO2007/042504 and WO2020/030618 and/or WO2022/013454).

In one embodiment of the present disclosure, the contrast agent is an agent that includes dihydrogen [(±)-4-carboxy-5,8,11-tris(carboxymethyl)-1-phenyl-2-oxa-5,8,11-triazatridecan-13-oato(5-)]gadolinate(2-) (also referred to as gadobenic acid).

In one embodiment of the present disclosure, the contrast agent is an agent that includes tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate (also referred to as gadoquatrane) (see for example J. Lohrke et al.: Preclinical Profile of Gadoquatrane: A Novel Tetrameric, Macrocyclic High Relaxivity Gadolinium-Based Contrast Agent. Invest Radiol., 2022, 1, 57(10): 629-638; WO2016193190).

In one embodiment of the present disclosure, the contrast agent is an agent that comprises a Gd³⁺ complex of a compound of the formula (I)

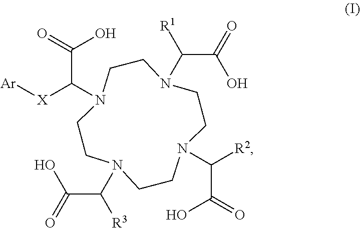

where
Ar is a group selected from

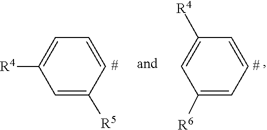

where _{#} is the linkage to X,
X is a group selected from
CH₂, (CH₂)₂, (CH₂)₃, (CH₂)₄ and *—(CH₂)₂O—CH₂—^{#},
where * is the linkage to Ar and ^{#} is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R is a hydrogen atom,
and
R⁶ is a hydrogen atom,
or a stereoisomer, tautomer, hydrate, solvate or salt thereof, or a mixture thereof.

In one embodiment of the present disclosure, the contrast agent is an agent that comprises a Gd³⁺ complex of a compound of the formula (II)

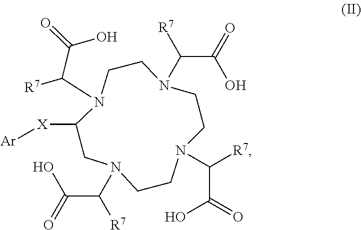

where
Ar is a group selected from

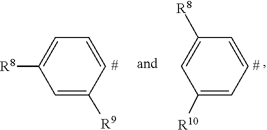

where _{#} is the linkage to X,
X is a group selected from CH₂, (CH₂)₂, (CH₂)₃, (CH₂)₄ and *—(CH₂)₂O—CH₂—^{#}, where * is the linkage to Ar and ^{#} is the linkage to the acetic acid residue,
R⁷ is a hydrogen atom or a group selected from C₁-C₃ alkyl, —CH₂OH,
—(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

The term “C₁-C₃ alkyl” denotes a linear or branched, saturated monovalent hydrocarbon group having 1, 2 or 3 carbon atoms, for example methyl, ethyl, n-propyl or isopropyl. The term “C₂-C₄ alkyl” denotes a linear or branched, saturated monovalent hydrocarbon group having 2, 3 or 4 carbon atoms.

The term “C₂-C₄ alkoxy” denotes a linear or branched, saturated monovalent group of the formula (C₂-C₄ alkyl)-O—, in which the term “C₂-C₄ alkyl” is as defined above, for example a methoxy, ethoxy, n-propoxy or isopropoxy group.

In one embodiment of the present disclosure, the contrast agent is an agent that includes gadolinium 2,2′,2″-(10-{1-carboxy-2-[2-(4-ethoxyphenyl)ethoxy]ethyl}-1,4,7,10-tetraazacyclododecane-1,4,7-triyl)triacetate (see for example WO2022/194777, example 1).

In one embodiment of the present disclosure, the contrast agent is an agent that includes gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 2).

In one embodiment of the present disclosure, the contrast agent is an agent that includes gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 4).

In one embodiment of the present disclosure, the contrast agent is an agent that includes gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate) (see for example WO2022/194777, example 15).

In one embodiment of the present disclosure, the contrast agent is an agent that includes gadolinium 2,2′,2″-{10-[(1S)-4-(4-butoxyphenyl)-1-carboxybutyl]-1,4,7,10-tetraazacyclododecane-1,4,7-triyl}triacetate (see for example WO2022/194777, example 31).

In one embodiment of the present disclosure, the contrast agent is an agent that includes gadolinium 2,2′,2″-{(2S)-10-(carboxymethyl)-2-[4-(2-ethoxyethoxy)benzyl]-1,4,7,10-tetraazacyclododecane-1,4,7-triyl}triacetate.

In one embodiment of the present disclosure, the contrast agent is an agent that includes gadolinium 2,2′,2″-[10-(carboxymethyl)-2-(4-ethoxybenzyl)-1,4,7,10-tetraazacyclododecane-1,4,7-triyl]triacetate.

In one embodiment of the present disclosure, the contrast agent is an agent that includes gadolinium(III) 5,8-bis(carboxylatomethyl)-2-[2-(methylamino)-2-oxoethyl]-10-oxo-2,5,8,11-tetraazadodecane-1-carboxylate hydrate (also referred to as gadodiamide).

In one embodiment of the present disclosure, the contrast agent is an agent that includes gadolinium(III) 2-[4-(2-hydroxypropyl)-7,10-bis(2-oxido-2-oxoethyl)-1,4,7,10-tetrazacyclododec-1-yl]acetate (also referred to as gadoteridol).

In one embodiment of the present disclosure, the contrast agent is an agent that includes gadolinium(III) 2,2′,2″-(10-((2R,3S)-1,3,4-trihydroxybutan-2-yl)-1,4,7,10-tetraazacyclododecane-1,4,7-triyl)triacetate (also referred to as gadobutrol or Gd-DO3A-butrol).

In a first step, the first representation and the second representation are received or generated.

The term “receiving” encompasses both the retrieving of representations and the accepting of representations transmitted for example to the computer system of the present disclosure. The representations may be received from a computed tomography system, from a magnetic resonance imaging system or from an ultrasound scanner. The radiological images may be read from one or more data memories and/or transmitted from a separate computer system.

Radiological images resulting from radiological examinations (especially in the case of computed tomography and sonography) are normally obtained as representations in real space (also called image space). The first representation and the second representation that are received or generated in a first step are representations in real space.

“Real space” is the ordinary three-dimensional Euclidean space that corresponds with the space which we humans experience with our senses and in which we move. A representation in real space is therefore the familiar representation for people.

In a representation in real space, also referred to in this description as real-space depiction or real-space representation, the examination region is normally represented by a large number of image elements (pixels or voxels) that may for example be in a raster arrangement, in which case each image element represents a part of the examination region and each image element may be assigned a color value or gray value. A format widely used in radiology for storing and processing representations in real space is the DICOM format. DICOM (Digital Imaging and Communications in Medicine) is an open standard for storing and exchanging information in medical image data management. Other formats for storing, processing and/or displaying representations in real space are also possible.

It is possible to carry out co-registration of representations in real space. “Co-registration” (also known in the prior art as “image registration”) is employed to bring two or more real-space depictions of the same examination region into the best possible alignment with one another. One of the real-space depictions is defined as the reference image, the other is termed the object image. In order to optimally fit this to the reference image, a compensating transformation is calculated.

On the basis of the first representation and the second representation, a third representation is generated.

The third representation represents at least part of the examination region of the examination object, likewise in real space.

The third representation represents the signal enhancement brought about in the examination region by the second amount of contrast agent. In other words, the third representation comprises the differences in the second representation compared to the first representation that are brought about by the second amount of contrast agent.

In a preferred embodiment, the generation of the third representation comprises a subtraction of the first representation from the second representation. In other words, in a preferred embodiment the third representation is the difference between the first and the second representation.

In a further step, a fourth representation is generated by combining the first representation with the third representation. Such a combination transfers to the first representation information about the contrast enhancement brought about by the second amount of the contrast agent in the examination region.

The combination may, for example, be or comprise addition of the third representation to the first representation α times, α being a positive or negative real number. In other words, the third representation is multiplied by a gain factor α and the multiplication result is added to the first representation. The mathematical methods (subtraction, addition, multiplication) normally concern the tonal values of the individual image elements (pixels, voxels).

The gain factor may be chosen by a user, i.e. may be variable, or may be predefined, i.e. may be predetermined.

The gain factor indicates the extent to which contrast is enhanced in the fourth representation.

The fourth representation can be output (e.g., displayed on a screen or printed using a printer), stored on a data memory and/or transmitted to a separate computer system.

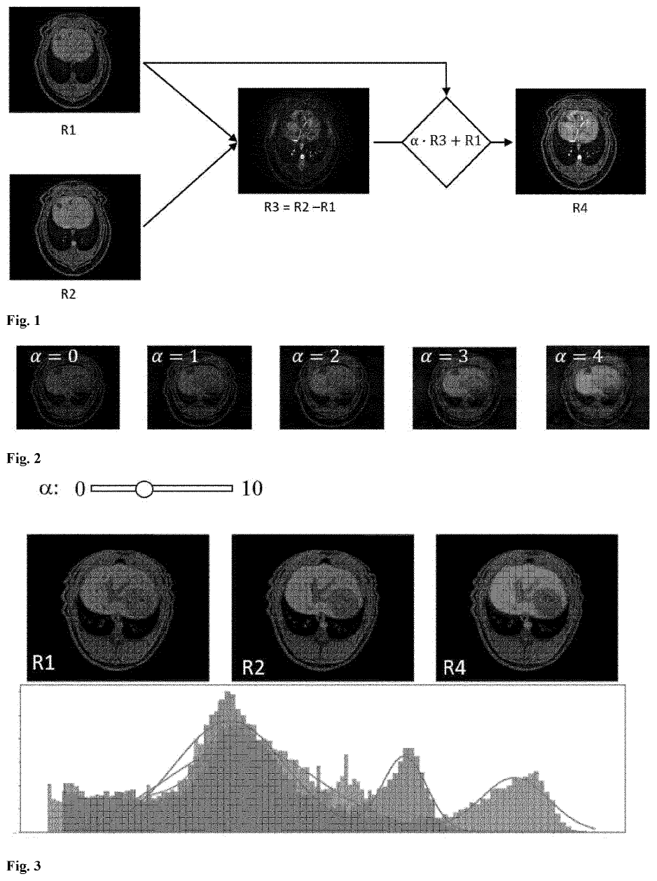

FIG. 1 shows schematically, in the form of an example, the generation of a third representation and a fourth representation on the basis of a first representation and a second representation.

FIG. 1 shows an examination region of an examination object in the form of various representations.

A first representation R1 represents the examination region in real space without contrast agent. The examination region shown in FIG. 1 includes a liver of a pig. The first representation R1 is a magnetic resonance image.

A second representation R2 represents the same examination region of the same examination object as the first representation R1 in real space. The second representation R2 is likewise a magnetic resonance image.

The second representation R2 represents the examination region after administration of an amount of a contrast agent. In the present example, an amount of 25 mol per kg body weight of a hepatobiliary contrast agent was administered intravenously to the examination object. The second representation R2 represents the examination region in the so-called arterial phase.

A hepatobiliary contrast agent has the characteristic features of being specifically taken up by liver cells (hepatocytes), accumulating in the functional tissue (parenchyma) and enhancing contrast in healthy liver tissue. An example of a hepatobiliary contrast agent is the disodium salt of gadoxetic acid (Gd-EOB-DTPA disodium), which is described in U.S. Pat. No. 6,039,931A and is commercially available under the trade names Primovist® and Eovist®. Further hepatobiliary contrast agents are described, inter alia, in WO2022/194777.

On the basis of the first representation R1 and the second representation R2, a third representation R3 is generated. In the example shown in FIG. 1, the third representation R3 is the difference between the second representation R2 and the first representation R1 (R3=R2−R1).

In the third representation, negative tonal values that can arise when subtracting the first representation from the second representation can be set to zero (or to another value) to avoid negative values.

The third representation R3 represents the contrast enhancement brought about in the examination region by the second amount of the contrast agent.

The third representation R3 is multiplied by the gain factor α and the multiplication result is added to the first representation R1. This generates a fourth representation R4. In the example shown in FIG. 1, the gain factor α=3, i.e. the third representation R3 is added to the first representation R1 three times.

The fourth representation R4 may be subjected to a normalization, that is to say the tonal values may be multiplied by a factor such that the tonal value having the highest value is represented for example by the hue “white” and the tonal value having the lowest value is represented for example by the hue “black”.

It is also possible to choose a gain factor of less than 1, i.e. the contrast between areas with contrast agent and areas without contrast agent is then lower in the fourth representation than in the second representation.

It is also possible to further reduce the contrast by negative values of the gain factor α. This may be useful, for example, for generating a representation of the examination region without contrast agent on the basis of a first representation representing the examination region with a first amount of a contrast agent and a second representation representing the examination region with a second amount of the contrast agent. For example, if the second amount is double the first amount, a gain factor of α=−1 results in a fourth representation that looks as if no contrast agent had been administered, provided that the signal enhancement brought about by the contrast agent increases linearly with the amount and the first and the second representation represent the examination region at the same time interval from the administration of the first/second amount.

It is also possible to choose negative a values such that areas of the examination region that experience a contrast agent-induced signal enhancement in the representation generated by measurement are completely dark (black) in the artificially generated representations.

It is likewise possible to achieve an enhancement that is greater than the contrast enhancement brought about by a standard amount of contrast agent. Such a contrast enhancement is not possible with the method described in WO2019/074938A1, unless training data is generated by administering people with an amount of contrast agent that is higher than the standard amount and thus outside the range approved by regulatory authorities.

FIG. 2 shows by way of example and in schematic form various representations of an examination region of an examination object. The representations differ by the gain factor α, which can assume values of 0, 1, 2, 3, and 4 in the present examples.

A gain factor of α=0 means that no contrast enhancement is performed in the first representation.

A gain factor of α=1 means that the third representation is added once to the first representation. The contrast enhancement is equivalent to that of the second representation.

A gain factor of α=2, 3 or 4 means that the third representation is added twice, three times or four times to the first representation. The contrast enhancement rises as the gain factor increases.

In the example shown in FIG. 2, an integer multiple of the third representation is in all cases added to the first representation. It is of course also possible to add a non-integer proportion of the third representation to the first representation (for example α=1.5 or α=3.7 or α=4.159). This means that an enhancement can be increased in a continuous manner.

It is also possible to determine the gain factor in an automated manner. It is for example possible to define at least one area in a depiction of the first and/or the second representation (for example on a screen) and/or to have it selected by a user and to set the gain factor such that a gray value in the depiction (or a different tonal value in the case of a depiction other than a gray-value depiction) assumes a defined value and/or is above or below a threshold value and/or that two gray values in two different selected or defined areas are at a defined distance from one another and/or are at a distance from one another that is above or below a threshold value. It is also possible to employ other criteria in the automated determination of the gain factor. The criteria for the automated determination of the gain factor may for example be based on the histogram of the first, second, third and/or fourth representation. Such a histogram may show the number of image elements having a defined tonal value or gray value.

FIG. 3 shows a preferred embodiment of an output of the artificial contrast-enhanced radiological image of an examination region by means of a computer system/computer program. The output is made to a user of the computer system and/or computer program of the present disclosure.

A first representation R1 of an examination region of an examination object, a second representation R2 of the examination region of the examination object and a fourth representation R4 of the examination region of the examination object are displayed to the user (for example on a monitor).

The first representation R1 represents the examination region without contrast agent or after administration of a first amount of a contrast agent.

The second representation R2 represents the examination region after administration of a second amount of the contrast agent. The second amount is larger than the first amount.

The fourth representation R4 represents the examination region with enhanced contrast. The contrast between areas without contrast agent and areas with contrast agent is greater in the case of the fourth representation R4 than in the second representation R2.

The fourth representation R4 was generated as described in this disclosure (see in particular the descriptions relating to FIG. 1).

Below the displayed representations R1, R2 and R4, the histograms of the representations are displayed to the user as superimposed histograms.

Above the displayed representations R1, R2 and R4, the user is provided with a virtual slider with which the user can make adjustments. Said slider allows the user to freely choose the gain factor α in a range from 1 to 10. The slider indicates that the gain factor can be increased from 1 to 10 in a continuous manner.

The output shown in FIG. 3 is preferably configured such that the display of the fourth representation R4 is immediately updated when the user makes changes by means of the slider. The user can then for example alter the gain factor α and immediately see how a change in the gain factor affects the representation R4. This allows the user to identify the settings that result in an optimal fourth representation R4 of the examination region for the user.

Any change to the parameter α results in the computer system using the altered parameter for background recalculation and display of the fourth representation R4. The same also applies to the histogram of the fourth representation R4.

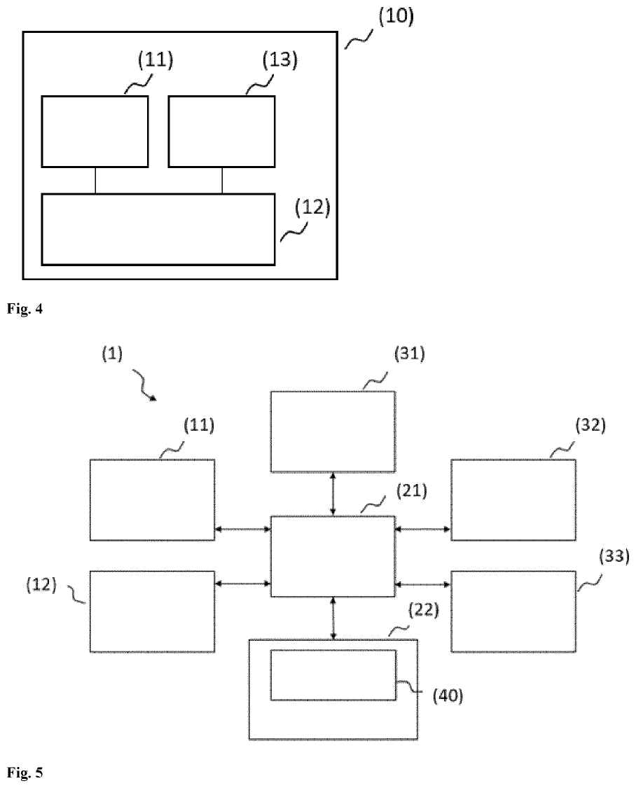

FIG. 4 shows by way of example and in schematic form a computer system according to the present disclosure.

A “computer system” is an electronic data processing system that processes data by means of programmable calculation rules. Such a system typically comprises a “computer”, which is the unit that comprises a processor for carrying out logic operations, and peripherals.

In computer technology, “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

The computer system (1) shown in FIG. 4 comprises an input unit (10), a control and calculation unit (20) and an output unit (30).

The control and calculation unit (20) is used for control of the computer system (1), for coordination of the data flows between the units of the computer system (1), and for the performance of calculations.

The control and calculation unit (20) is configured:
to cause the receiving unit to receive a first representation, the first representation representing an examination region of an examination object without contrast agent or after administration of a first amount of a contrast agent,
to cause the receiving unit to receive a second representation, the second representation representing the examination region of the examination object after administration of a second amount of the contrast agent,
to generate a third representation on the basis of the first representation and the second representation, the generating the third representation comprising subtracting the first representation from the second representation,
to generate a fourth representation on the basis of the first representation and the third representation, the generating the fourth representation comprising adding the third representation to the first representation α times, α being a positive or negative real number,
to cause the output unit to output the fourth representation and/or to store it and/or to transmit it to a separate computer system.

FIG. 5 schematically shows by way of example a further embodiment of the computer system.

The computer system (1) comprises a processing unit (21) connected to a memory (22). The processing unit (21) and the memory (22) form a control and calculation unit, as shown in FIG. 4.

The processing unit (21) may comprise one or more processors alone or in combination with one or more memories. The processing unit (21) may be standard computer hardware capable of processing information such as digital images, computer programs, and/or other digital information. The processing unit (21) typically consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the memory (22) of the same or of a different computer system.

The memory (22) may be customary computer hardware that is able to store information such as digital images (for example representations of the examination region), data, computer programs and/or other digital information either temporarily and/or permanently. The memory (22) may comprise a volatile and/or nonvolatile memory and may be nonremovable or removable. Examples of suitable memories are RAM (random access memory), ROM (read-only memory), a hard disk, a flash memory, an exchangeable computer floppy disk, an optical disk, a magnetic tape or a combination of the aforementioned. Optical disks may include compact disks with read-only memory (CD-ROM), compact disks with read/write function (CD-R/W), DVDs, Blu-ray disks, and the like.

The processing unit (21) may be connected not only to the memory (22) but also to one or more interfaces (11, 12, 31, 32, 33) in order to display, transmit and/or receive information. The interfaces may comprise one or more communication interfaces (32, 33) and/or one or more user interfaces (11, 12, 31). The one or more communication interfaces may be configured to send and/or receive information, for example to and/or from an MRI scanner, a CT scanner, an ultrasound camera, other computer systems, networks, data memories or the like. The one or more communication interfaces may be configured to transmit and/or receive information via physical (wired) and/or wireless communication connections. The one or more communication interfaces may comprise one or more interfaces for connection to a network, for example using technologies such as cellphone, Wi-Fi, satellite, cable, DSL, optical fiber, and/or the like. In some examples, the one or more communication interfaces may comprise one or more close-range communication interfaces configured to connect devices with short-range communication technologies such as NFC, RFID, Bluetooth, Bluetooth LE, ZigBee, infrared (e.g. IrDA) or the like.

The user interfaces may comprise a display (31). A display (31) may be configured to display information to a user. Suitable examples thereof are a liquid-crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces are a microphone, an image or video recording device (for example a camera), a keyboard or a keypad, a joystick, a touch-sensitive surface (separate from a touchscreen or integrated therein) or the like. In some examples, the user interfaces may contain an automatic identification and data capture technology (AIDC) for machine-readable information. This may include barcodes, radiofrequency identification (RFID), magnetic strips, optical character recognition (OCR), integrated circuit cards (ICC) and the like. The user interfaces may also include one or more interfaces for communication with peripherals such as printers and the like.

One or more computer programs (40) may be stored in the memory (22) and executed by the processing unit (21), which is thereby programmed to fulfill the functions described in this description. The retrieving, loading, and execution of instructions in the computer program (40) may take place sequentially, such that an instruction is respectively retrieved, loaded and executed. However, the retrieving, loading and/or execution may also take place in parallel.

The computer system of the present disclosure may be designed as a laptop, notebook, netbook and/or tablet PC; it may also be a component of an MRI scanner, a CT scanner or an ultrasound diagnostic device.

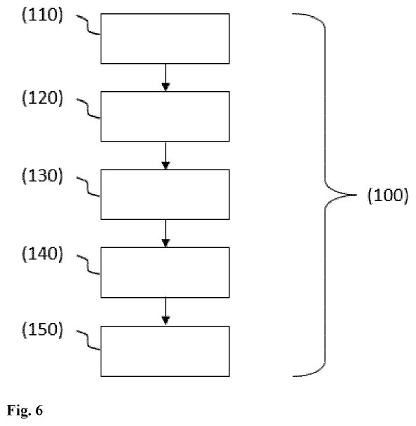

FIG. 6 shows by way of example and in schematic form an embodiment of the computer-implemented method in the form of a flowchart.

The method (100) comprises the following steps:
(110) receiving or generating a first representation, the first representation representing an examination region of an examination object without contrast agent or after administration of a first amount of a contrast agent,
(120) receiving or generating a second representation, the second representation representing the examination region of the examination object after administration of a second amount of the contrast agent, the second amount being larger than the first amount,
(130) generating a third representation on the basis of the first representation and the second representation, the generating the third representation comprising subtracting the first representation from the second representation,
(140) generating a fourth representation, the generating the fourth representation comprising adding the third representation to the first representation α times, α being a positive or negative real number,
(150) outputting and/or storing the fourth representation and/or transmitting the fourth representation of the examination region to a separate computer system.

The present invention can be used for various purposes. Some examples of use are described below, without any intention of limiting the invention to these examples of use.

A first example of use concerns magnetic resonance imaging examinations for differentiating intraaxial tumors such as intracerebral metastases and malignant gliomas. The infiltrative growth of these tumors makes it difficult to differentiate exactly between tumor and healthy tissue. Determining the extent of a tumor is however crucial for surgical removal. Distinguishing between tumors and healthy tissue is facilitated by administration of an extracellular MRI contrast agent; after intravenous administration of a standard dose of 0.1 mmol/kg body weight of the extracellular MRI contrast agent gadobutrol, intraaxial tumors can be differentiated much more readily. At higher doses, the contrast between lesion and healthy brain tissue is increased further; the detection rate of brain metastases increases linearly with the dose of the contrast agent (see for example M. Hartmann et al.: Does the administration of a high dose of a paramagnetic contrast medium (Gadovist) improve the diagnostic value of magnetic resonance tomography in glioblastomas?doi: 10.1055/s-2007-1015623).

A single triple dose or a second subsequent dose may be administered here up to a total dose of 0.3 mmol/kg body weight. This exposes the patient and the environment to additional gadolinium and in the case of a second scan, incurs further additional costs.

The present invention can be used to avoid a dose of contrast agent exceeding the standard amount. A first MRI image can be generated without contrast agent or with an amount less than the standard amount and a second MRI image generated with the standard amount. On the basis of these generated MRI images it is possible, as described in this disclosure, to generate a synthetic MRI image in which the contrast between lesions and healthy tissue can be varied within wide limits by altering the gain factor α. This makes it possible to achieve contrasts that are otherwise achievable only by administering an amount of contrast agent larger than the standard amount.

Another example of use concerns the reduction of the amount of MRI contrast agent in a magnetic resonance imaging examination. Gadolinium-containing contrast agents such as gadobutrol are used for a multitude of examinations. They are used for contrast enhancement in skull examinations, spine examinations, breast examinations or other examinations. In the central nervous system, gadobutrol emphasizes regions where the blood-brain barrier is impaired and/or vessels are abnormal. In breast tissue, gadobutrol visualizes the presence and extent of malignant breast disease. Gadobutrol is also used in contrast-enhanced magnetic resonance angiography for diagnosing stroke, for detecting tumor blood perfusion, and for detecting focal cerebral ischemia.

Owing to the increasing impact on the environment, to the cost burden falling on healthcare systems, and to concerns about acute side effects and possible long-term health risks, especially in the case of repeated and long-term exposure, a reduction in the dose of gadolinium-containing contrast agents is desired. This can be achieved by the present invention.

A first MRI image without contrast agent and a second MRI image with an amount of contrast agent less than the standard amount can be generated. On the basis of these generated MRI images it is possible, as described in this disclosure, to generate a synthetic MRI image in which the contrast can be varied within wide limits by altering the gain factor α. This makes it possible with less than the standard amount of contrast agent to achieve the same contrast as is obtained after administration of the standard amount.

Another example of use concerns the detection, identification and/or characterization of lesions in the liver with the aid of a hepatobiliary contrast agent such as Primovist®.

Primovist® is administered intravenously (i.v.) at a standard dose of 0.025 mmol/kg body weight. This standard dose is lower than the standard dose of 0.1 mmol/kg body weight in the case of extracellular MRI contrast agents. Unlike in contrast-enhanced MRI with extracellular gadolinium-containing contrast agents, Primovist® permits dynamic multiphase T1w imaging. However, the lower dose of Primovist® and the observation of transient motion artefacts that can occur shortly after intravenous administration means that contrast enhancement with Primovist® in the arterial phase is perceived by radiologists as poorer than contrast enhancement with extracellular MRI contrast agents. The assessment of the contrast enhancement in the arterial phase and of the vascularity of focal liver lesions is however of critical importance for accurate characterization of the lesion.

With the aid of the present invention it is possible to increase contrast, particularly in the arterial phase, without the need to administer a higher dose.

A first MRI image without contrast agent and a second MRI image during the arterial phase after administering an amount of a contrast agent that corresponds to the standard amount can be generated. On the basis of these generated MRI images it is possible, as described in this disclosure, to generate a synthetic MRI image in which the contrast in the arterial phase can be varied within wide limits by altering the gain factor α. This makes it possible to achieve contrasts that are otherwise achievable only by administering an amount of contrast agent larger than the standard amount.

Another example of use concerns the use of MRI contrast agents in computed tomography examinations.

In a CT examination, MRI contrast agents usually have a lower contrast-enhancing effect than CT contrast agents. However, it can be advantageous to employ an MRI contrast agent in a CT examination.

An example is a minimally invasive intervention in the liver of a patient, where a surgeon is monitoring the procedure by means of a CT scanner. Computed tomography (CT) has the advantage over magnetic resonance imaging that more major surgical interventions are possible in an examination region of an examination object while generating CT images of the examination region. By contrast, there are only few surgical instruments and surgical devices that are MRI-compatible. Moreover, access to the patient is restricted by the magnets used in MRI. Thus, while performing a procedure in the examination region, a surgeon will be able to visualize the examination region by CT and to follow the procedure on a monitor.

For example, if a surgeon wishes to perform a procedure in a patient's liver in order for example to carry out a biopsy on a liver lesion or to remove a tumor, the contrast between a liver lesion or tumor and healthy liver tissue will not be as pronounced in a CT image of the liver as it is in an MRI image after administration of a hepatobiliary contrast agent. There are currently no known and/or authorized CT-specific hepatobiliary contrast agents in CT. The use of an MRI contrast agent, more particularly a hepatobiliary MRI contrast agent, in computed tomography thus combines the possibility of differentiating between healthy and diseased liver tissue and the possibility of carrying out an operation with simultaneous visualization of the liver.

The comparatively low contrast enhancement achieved by the MRI contrast agent can be increased with the aid of the present invention without the need to administer a dose higher than the standard dose.

A first CT image without MRI contrast agent and a second CT image after administering an amount of an MRI contrast agent that corresponds to the standard amount can be generated. On the basis of these generated CT images it is possible, as described in this disclosure, to generate a synthetic CT image in which the contrast produced by the MRI contrast agent can be varied within wide limits by altering the gain factor α. This makes it possible to achieve contrasts that are otherwise achievable only by administering an amount of MRI contrast agent larger than the standard amount.

1. A computer-implemented method comprising:
receiving a first representation, the first representation representing an examination region of an examination object without contrast agent or after administration of a first amount of a contrast agent,
receiving a second representation, the second representation representing the examination region of the examination object after administration of a second amount of the contrast agent, the second amount being larger than the first amount,
generating a third representation, the generating the third representation comprising subtracting the first representation from the second representation,
generating a fourth representation, the generating the fourth representation comprising adding the third representation to the first representation α times, α being a positive or negative real number,
outputting and/or storing the fourth representation and/or transmitting the fourth representation to a separate computer system.

2. The method as claimed in claim 1, wherein the examination object is a living being, preferably a mammal, most preferably a human.

3. The method as claimed in claim 1 or 2, wherein the examination region includes a liver, kidney, heart, lung, brain, stomach, bladder, prostate, intestine and/or a part thereof and/or another/further part of the body of a human.

4. The method as claimed in any of claims 1 to 3, wherein α is greater than 1.

5. The method as claimed in any of claims 1 to 4, further comprising:
receiving one or more values for a from a user.

6. The method as claimed in any of claims 1 to 5, further comprising:
receiving a first tonal value of a first image element of a depiction of the first representation or the second representation,
receiving a second tonal value of a second image element of a depiction of the first representation or the second representation,
determining a value for a for which the difference between the first tonal value and the second tonal value assumes a predefined value or is above or below a predefined threshold value.

7. The method as claimed in any of claims 1 to 6, wherein the first representation and the second representation are the result of a magnetic resonance imaging examination and/or have been generated from magnetic resonance images.

8. The method as claimed in any of claims 1 to 7, wherein the contrast agent comprises one of the following substances:
gadolinium(III) 2-[4,7,10-tris(carboxymethyl)-1,4,7,10-tetrazacyclododec-1-yl]acetic acid,
gadolinium(III) ethoxybenzyldiethylenetriaminepentaacetic acid,
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate,
dihydrogen [(±)-4-carboxy-5,8,11-tris(carboxymethyl)-1-phenyl-2-oxa-5,8,11-triazatridecan-13-oato(5-)]gadolinate(2-),
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate,
2,2′,2″-(10-{1-carboxy-2-[2-(4-ethoxyphenyl)ethoxy]ethyl}-1,4,7,10-tetraazacyclododecane-1,4,7-triyl)triacetate,
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate,
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate,
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate)
gadolinium 2,2′,2″-{10-[(1S)-4-(4-butoxyphenyl)-1-carboxybutyl]-1,4,7,10-tetraazacyclododecane-1,4,7-triyl}triacetate,
gadolinium(III) 5,8-bis(carboxylatomethyl)-2-[2-(methylamino)-2-oxoethyl]-10-oxo-2,5,8,11-tetraazadodecane-1-carboxylate hydrate
gadolinium(III) 2-[4-(2-hydroxypropyl)-7,10-bis(2-oxido-2-oxoethyl)-1,4,7,10-tetrazacyclododec-1-yl]acetate,
gadolinium(III) 2,2′,2″-(10-((2R,3S)-1,3,4-trihydroxybutan-2-yl)-1,4,7,10-tetraazacyclododecane-1,4,7-triyl)triacetate,
gadolinium 2,2′,2″-{(2S)-10-(carboxymethyl)-2-[4-(2-ethoxyethoxy)benzyl]-1,4,7,10-tetraazacyclododecane-1,4,7-triyl}triacetate,
gadolinium 2,2′,2″-[10-(carboxymethyl)-2-(4-ethoxybenzyl)-1,4,7,10-tetraazacyclododecane-1,4,7-triyl]triacetate.

9. A computer system comprising
a processor; and
a memory that stores an application program configured to perform an operation when executed by the processor, said operation comprising:
receiving a first representation, the first representation representing an examination region of an examination object without contrast agent or after administration of a first amount of a contrast agent,
receiving a second representation, the second representation representing the examination region of the examination object after administration of a second amount of the contrast agent, the second amount being larger than the first amount,
generating a third representation on the basis of the first representation and the second representation, the generating the third representation comprising subtracting the first representation from the second representation,
generating a fourth representation, the generating the fourth representation comprising adding the third representation to the first representation α times, α being a positive or negative real number,
outputting and/or storing the fourth representation and/or transmitting the fourth representation to a separate computer system.

10. A computer program product comprising a data carrier on which a computer program is stored, the computer program being loadable into a working memory of a computer system, where it causes the computer system to execute the following steps:
receiving a first representation, the first representation representing an examination region of an examination object without contrast agent or after administration of a first amount of a contrast agent,
receiving a second representation, the second representation representing the examination region of the examination object after administration of a second amount of the contrast agent, the second amount being larger than the first amount,
generating a third representation on the basis of the first representation and the second representation, the generating the third representation comprising subtracting the first representation from the second representation,
generating a fourth representation, the generating the fourth representation comprising adding the third representation to the first representation α times, α being a positive or negative real number,
outputting and/or storing the fourth representation and/or transmitting the fourth representation to a separate computer system.

11. The use of a contrast agent in a radiological examination method comprising:
receiving or generating a first representation, the first representation representing an examination region of an examination object without the contrast agent or after administration of a first amount of the contrast agent,
receiving or generating a second representation, the second representation representing the examination region of the examination object after administration of a second amount of the contrast agent, the second amount being larger than the first amount,
generating a third representation on the basis of the first representation and the second representation, the generating the third representation comprising subtracting the first representation from the second representation,
generating a fourth representation, the generating the fourth representation comprising adding the third representation to the first representation α times, α being a positive or negative real number,
outputting and/or storing the fourth representation and/or transmitting the fourth representation to a separate computer system.

12. The use as claimed in claim 11, wherein the radiological examination method is a magnetic resonance imaging examination and wherein the contrast agent comprises a Gd³⁺ complex of a compound of the formula (I) 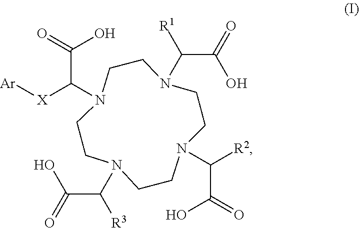
where
Ar is a group selected from 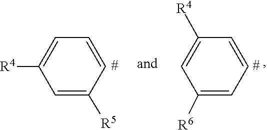
where _{#} is the linkage to X,
X is a group selected from
CH₂, (CH₂)₂, (CH₂)₃, (CH₂)₄ and *—(CH₂)₂O—CH₂—^{#},
where * is the linkage to Ar and ^{#} is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof.

13. The use as claimed in claim 11, wherein the radiological examination method is a magnetic resonance imaging examination and wherein the contrast agent comprises a Gd³⁺ complex of a compound of the formula (II) 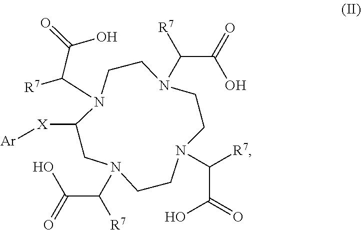
where
Ar is a group selected from 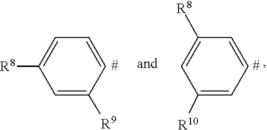
where _{#} is the linkage to X,
X is a group selected from CH₂, (CH₂)₂, (CH₂)₃, (CH₂)₄ and *—(CH₂)₂O—CH₂—^{#}, where * is the linkage to Ar and ^{#} is the linkage to the acetic acid residue,
R⁷ is a hydrogen atom or a group selected from C₁-C₃ alkyl, —CH₂OH,
—(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

14. The use as claimed in claim 11, wherein the radiological examination method is a magnetic resonance imaging examination and wherein the contrast agent comprises one of the following substances:
gadolinium(III) 2-[4,7,10-tris(carboxymethyl)-1,4,7,10-tetrazacyclododec-1-yl]acetic acid,
gadolinium(III) ethoxybenzyldiethylenetriaminepentaacetic acid,
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate,
dihydrogen [(±)-4-carboxy-5,8,11-tris(carboxymethyl)-1-phenyl-2-oxa-5,8,11-triazatridecan-13-oato(5-)]gadolinate(2-),
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate,
2,2′,2″-(10-{1-carboxy-2-[2-(4-ethoxyphenyl)ethoxy]ethyl}-1,4,7,10-tetraazacyclododecane-1,4,7-triyl)triacetate,
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate,
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate,
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate)
gadolinium 2,2′,2″-{10-[(1S)-4-(4-butoxyphenyl)-1-carboxybutyl]-1,4,7,10-tetraazacyclododecane-1,4,7-triyl}triacetate,
gadolinium(III) 5,8-bis(carboxylatomethyl)-2-[2-(methylamino)-2-oxoethyl]-10-oxo-2,5,8,11-tetraazadodecane-1-carboxylate hydrate
gadolinium(III) 2-[4-(2-hydroxypropyl)-7,10-bis(2-oxido-2-oxoethyl)-1,4,7,10-tetrazacyclododec-1-yl]acetate,
gadolinium(III) 2,2′,2″-(10-((2R,3S)-1,3,4-trihydroxybutan-2-yl)-1,4,7,10-tetraazacyclododecane-1,4,7-triyl)triacetate,
gadolinium 2,2′,2″-{(2S)-10-(carboxymethyl)-2-[4-(2-ethoxyethoxy)benzyl]-1,4,7,10-tetraazacyclododecane-1,4,7-triyl}triacetate,
gadolinium 2,2′,2″-[10-(carboxymethyl)-2-(4-ethoxybenzyl)-1,4,7,10-tetraazacyclododecane-1,4,7-triyl]triacetate.

15. A contrast agent for use in a radiological examination method comprising:
receiving or generating a first representation, the first representation representing an examination region of an examination object without the contrast agent or after administration of a first amount of the contrast agent,
receiving or generating a second representation, the second representation representing the examination region of the examination object after administration of a second amount of the contrast agent, the second amount being larger than the first amount,
generating a third representation on the basis of the first representation and the second representation, the generating the third representation comprising subtracting the first representation from the second representation,
generating a fourth representation, the generating the fourth representation comprising adding the third representation to the first representation α times, α being a positive or negative real number,
outputting and/or storing the fourth representation and/or transmitting the fourth representation to a separate computer system.

16. The contrast agent for use as claimed in claim 15, wherein the radiological examination method is a magnetic resonance imaging examination and wherein the contrast agent comprises a Gd³⁺ complex of a compound of the formula (I) 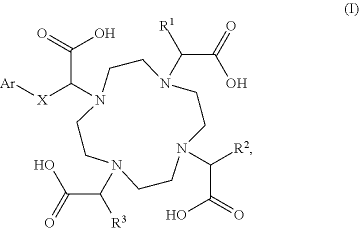
where
Ar is a group selected from 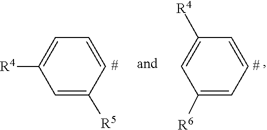
where _{#} is the linkage to X,
X is a group selected from
CH₂, (CH₂)₂, (CH₂)₃, (CH₂)₄ and *—(CH₂)₂O—CH₂—^{#},
where * is the linkage to Ar and ^{#} is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof.

17. The contrast agent for use as claimed in claim 15, wherein the radiological examination method is a magnetic resonance imaging examination and wherein the contrast agent comprises a Gd³⁺ complex of a compound of the formula (II) 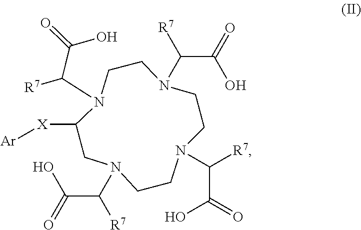
where
Ar is a group selected from 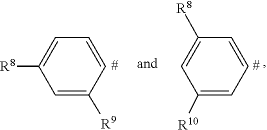
where _{#} is the linkage to X,
X is a group selected from CH₂, (CH₂)₂, (CH₂)₃, (CH₂)₄ and *—(CH₂)₂O—CH₂—^{#}, where * is the linkage to Ar and ^{#} is the linkage to the acetic acid residue,
R⁷ is a hydrogen atom or a group selected from C₁-C₃ alkyl, —CH₂OH,
—(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

18. The contrast agent for use as claimed in claim 15, wherein the contrast agent comprises one of the following substances:
gadolinium(III) 2-[4,7,10-tris(carboxymethyl)-1,4,7,10-tetrazacyclododec-1-yl]acetic acid,
gadolinium(III) ethoxybenzyldiethylenetriaminepentaacetic acid,
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate,
dihydrogen [(±)-4-carboxy-5,8,11-tris(carboxymethyl)-1-phenyl-2-oxa-5,8,11-triazatridecan-13-oato(5-)]gadolinate(2-),
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate,
2,2′,2″-(10-{1-carboxy-2-[2-(4-ethoxyphenyl)ethoxy]ethyl}-1,4,7,10-tetraazacyclododecane-1,4,7-triyl)triacetate,
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate,
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate,
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate)
gadolinium 2,2′,2″-{10-[(1S)-4-(4-butoxyphenyl)-1-carboxybutyl]-1,4,7,10-tetraazacyclododecane-1,4,7-triyl}triacetate,
gadolinium(III) 5,8-bis(carboxylatomethyl)-2-[2-(methylamino)-2-oxoethyl]-10-oxo-2,5,8,11-tetraazadodecane-1-carboxylate hydrate
gadolinium(III) 2-[4-(2-hydroxypropyl)-7,10-bis(2-oxido-2-oxoethyl)-1,4,7,10-tetrazacyclododec-1-yl]acetate,
gadolinium(III) 2,2′,2″-(10-((2R,3S)-1,3,4-trihydroxybutan-2-yl)-1,4,7,10-tetraazacyclododecane-1,4,7-triyl)triacetate,
gadolinium 2,2′,2″-{(2S)-10-(carboxymethyl)-2-[4-(2-ethoxyethoxy)benzyl]-1,4,7,10-tetraazacyclododecane-1,4,7-triyl}triacetate,
gadolinium 2,2′,2″-[10-(carboxymethyl)-2-(4-ethoxybenzyl)-1,4,7,10-tetraazacyclododecane-1,4,7-triyl]triacetate.

19. A kit comprising a computer program product as claimed in claim 10 and a contrast agent.

20. The kit as claimed in claim 19, wherein the contrast agent comprises a Gd³⁺ complex of a compound of the formula (I) 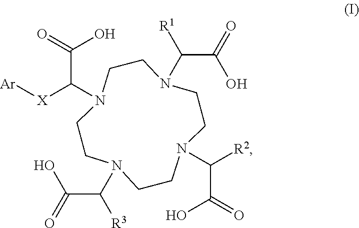
where
Ar is a group selected from 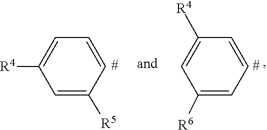
where _{#} is the linkage to X,
X is a group selected from
CH₂, (CH₂)₂, (CH₂)₃, (CH₂)₄ and *—(CH₂)₂O—CH₂—^{#},
where * is the linkage to Ar and ^{#} is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof.

21. The kit as claimed in claim 19, wherein the contrast agent comprises a Gd³⁺ complex of a compound of the formula (II) 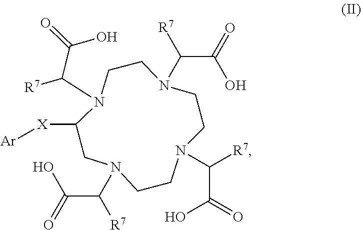
where
Ar is a group selected from 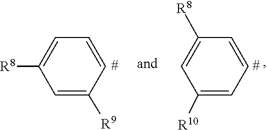
where _{#} is the linkage to X,
X is a group selected from CH₂, (CH₂)₂, (CH₂)₃, (CH₂)₄ and *—(CH₂)₂O—CH₂—^{#}, where * is the linkage to Ar and ^{#} is the linkage to the acetic acid residue,
R⁷ is a hydrogen atom or a group selected from C₁-C₃ alkyl, —CH₂OH,
—(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

22. The kit as claimed in claim 19, wherein the contrast agent comprises one of the following substances:
gadolinium(III) 2-[4,7,10-tris(carboxymethyl)-1,4,7,10-tetrazacyclododec-1-yl]acetic acid,
gadolinium(III) ethoxybenzyldiethylenetriaminepentaacetic acid,
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate,
dihydrogen [(±)-4-carboxy-5,8,11-tris(carboxymethyl)-1-phenyl-2-oxa-5,8,11-triazatridecan-13-oato(5-)]gadolinate(2-),
tetragadolinium [4,10-bis(carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate,
2,2′,2″-(10-{1-carboxy-2-[2-(4-ethoxyphenyl)ethoxy]ethyl}-1,4,7,10-tetraazacyclododecane-1,4,7-triyl)triacetate,
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate,
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate,
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate)
gadolinium 2,2′,2″-{10-[(1S)-4-(4-butoxyphenyl)-1-carboxybutyl]-1,4,7,10-tetraazacyclododecane-1,4,7-triyl}triacetate,
gadolinium(III) 5,8-bis(carboxylatomethyl)-2-[2-(methylamino)-2-oxoethyl]-10-oxo-2,5,8,11-tetraazadodecane-1-carboxylate hydrate
gadolinium(III) 2-[4-(2-hydroxypropyl)-7,10-bis(2-oxido-2-oxoethyl)-1,4,7,10-tetrazacyclododec-1-yl]acetate,
gadolinium(III) 2,2′,2″-(10-((2R,3S)-1,3,4-trihydroxybutan-2-yl)-1,4,7,10-tetraazacyclododecane-1,4,7-triyl)triacetate,
gadolinium 2,2′,2″-{(2S)-10-(carboxymethyl)-2-[4-(2-ethoxyethoxy)benzyl]-1,4,7,10-tetraazacyclododecane-1,4,7-triyl}triacetate,
gadolinium 2,2′,2″-[10-(carboxymethyl)-2-(4-ethoxybenzyl)-1,4,7,10-tetraazacyclododecane-1,4,7-triyl]triacetate.
